(19) United States
(12) Patent Application Publication
(10) Pub. No.: US 2020/0206374 A1
(43) Pub. Date: Jul. 2, 2020

(54) SUBSEA BIOFOULING PREVENTER DEVICE

(71) Applicant: Vetco Gray Scandinavia AS, Sandvika (NO)

(72) Inventors: Aymer Yeferson Maturana CORDOBA, Rio de Janeiro (BR); Bernardo Alves CINELLI, Rio de Janeiro (BR); Bruno BETONI PARODI, Rio de Janeiro (BR); Zamir ALAM, Oakville (CA); Sandro Guimaraes SOUZA, Rio de Janeiro (BR); Ana Carolina Miranda COSTA, Rio de Janeiro (BR); Hua WANG, Niskayuna, NY (US); Jimmy A. LOPEZ, Rio de Janeiro (BR)

(21) Appl. No.: 16/643,615

(22) PCT Filed: Aug. 23, 2018
(86) PCT No.: PCT/EP2018/072781

§ 371 Date: Mar. 2, 2020

(30) Foreign Application Priority Data
Aug. 29, 2017 (NO) 20171399

(51) Int. Cl. A61L 2/10; A61L 2/238; E21B 41/00; C09D 5/16; B01D 65/08

(52) U.S. Cl. CPC A61L 2/10; A61L 2/238; E21B 41/0007; B82Y 30/00; B01D 65/08; A61L 2202/20; A61L 2202/10; C09D 5/1618

(57) ABSTRACT

The present invention relates to a high-intensity ultraviolet light emitting diodes (6) (UV-LED) device (1) for preventing biofouling formation in a system for subsea operation of a target fluid. Further, the present invention discloses such device for coupling or integration into a subsea treatment system and to a process using such device.

### FIELD OF THE INVENTION

The present invention relates to a device for preventing biofouling formation in a system for subsea operation of a target fluid. The device includes high-intensity ultraviolet light emitting diodes (UV-LED). Further, the present invention discloses integration of the device into a membrane system and a process using such device.

### BACKGROUND OF THE INVENTION

In seawater treatment, oil-water separation and gas processing in offshore and subsea exploration areas (subsea factory concept) biofouling is a challenge. Because of high maintenance costs and complexity of associated operations, a significantly increased life span of equipment, such as membranes, in subsea treatment systems is one of the most demanding requirements. Chemical utilization combined with physical and mechanical methods is the most widely utilized strategy for membrane and equipment cleaning onshore and topside. For biofouling prevention during subsea application of membrane processes there are a number of challenges, limitations and gaps that do not make application of conventional disinfection or biofouling control methods like those used on the surface, simple or obvious. In the case of membrane processes, biofouling represents around 80% of the total fouling. Thus, biofouling control is critical to guarantee processes, devices, equipment and instruments life and performance. Significant efforts and technological developments are being made to improve offshore and subsea related processes, e.g. in membrane treatment and other technologies. Especially, for applications like sulphate removal, low salinity water for flooding, oil/water and gas/gas separation, etc., which are strongly associated with enhanced oil recovery, increased productivity, improved operating conditions or reduction of the environmental impacts/risk of oil and gas exploration in deep and ultra-deep waters are sought.

Chemical and most of the conventional biofouling prevention methods are technically and/or economically unfeasible for subsea application, because of the very high cost, very complex operation and challenging conditions involved in a subsea operation and environment. For biofouling prevention during application of membrane processes like microfiltration (MF), ultrafiltration (UF), nanofiltration (NF), reverse osmosis (RO) or ion exchange (IE), there are a number of factors making conventional disinfection methods unsuitable. Such factors include mechanical resistance, chemical stability, physical security, monitoring and control, pressure (300 bar or more) and low temperature resistance, and also with the obligation to prevent any possibility of leakage into the sea of any toxic, dangerous or potentially harmful substance. Further, the method or system used must also be robust, with an extra-long life span, and must be maintenance free.

The applicant is engaged in several research programs for seawater treatment, oil-water separation and gas processing in offshore and subsea exploration areas. In parallel, auxiliary equipment and technologies need to be developed to support the new proposed developments in subsea processing and their unknown potential consequences. Thus, for subsea environment, new cleaning techniques must be implemented, to remove or prevent biofouling, and overcoming the drawbacks with existing technology.

UV light is a proven technology for microorganism inactivation in membrane water systems and other applications. By way of example, US20100176056A1 is directed to a method for preventing biofouling on surfaces using ultraviolet pre-treatment. This is directed to use of conventional low pressure UV lamps, designed for onshore application only, and being unfeasible for subsea operation. KR100971499B1 is directed to an apparatus for seawater desalinating with reverse osmosis. As for US20100176056A1, this patent is also designed for onshore application. Further, this patent presents a coupled disinfection unit to a membrane system, which limits the utilization for other applications. Madaeni et al. (2007), Characterization of self-cleaning RO membranes coated with TiO2 particles under UV irradiation, Journal of Membrane Science, ScienceDirect, 303 (1-2), 221-233, discloses a mechanism which acts directly on a membrane surface, and not on a fluid. The action on the membrane surface could cause interference and performance reduction of the membrane. Also, the wave length of the conventional UV light emission source used to activate the TiO2 is 365 nm, that do not have germicidal effect. Thus, the disinfection effect could be only due to the hydroxyl radicals that are produced during a short period. Hence, the cell inactivation reach Log 1 or less only.

It is hence a need for alternative products and methods for preventing biofouling formation in subsea operations and equipment. It has surprisingly been found that the proposed concept using UV-LEDs meet the specific critical to quality parameters inherent to subsea operation, and provide several benefits.

### BRIEF SUMMARY OF THE INVENTION

In this invention, a high-intensity ultraviolet light emitting diodes (UV-LED) device and process are provided. The device comes with all elements needed to make it able for deep or ultra-deep water operation for preventing biofouling formation, for example for subsea operation of a target fluid in a system. The device can be used to protect, for example, any subsea membrane process. The main objective is to prevent biofouling formation and organic matter deposition and incrustation in any parts of the system involving the target fluid. The UV-LED device provides a way to extend equipment life spam without, or with reduced, chemical utilization, free of maintenance and reducing the very high costs and complexity involved in any subsea maintenance operation. Hence, in a first aspect the invention provides a device for biofouling prevention in a system, such as a subsea system, comprising a target fluid, the device comprising a reactor unit comprising reactor surfaces, wherein the surfaces comprise high intensity ultraviolet light emitting diodes (UV-LED). Hence, the invention provides a subsea biofouling preventer device comprising a reactor unit comprising reactor surfaces, wherein the surfaces comprise high intensity ultraviolet light emitting diodes (UV-LED).

Further, the device may be included in a fluid treatment system and the device hence includes means for integrating this with a system, such as with a membrane separation system.

Even further, the invention provides a process for subsea operation of a target fluid in a system, comprising a step wherein high intensity ultraviolet radiation from UV-LEDs transmits through the target fluid to be treated to prevent biofouling formation in any parts of the system.

### BRIEF DESCRIPTION OF THE DRAWINGS

FIG. 1 provides a sketch of a device of the invention, wherein the LED reactor has a cylindrical structure.

FIG. 2 provides a further sketch of a device of the invention, showing how the wall mounted LEDs of the cylindrical reactor irradiate towards the centre of the cylinder.

FIG. 3 provides sketches of a device of the invention wherein the device has a pipe-in-pipe configuration. In FIG. 3a the LEDs are placed on a side cover and in 3b the LEDs are placed also on the reactor walls of the outer pipe tank, either on the outer or inner walls.

FIGS. 4a and 4b provide sketches of how the wall mounted LEDs can be arranged in a cylindrical reactor, with two (a) or three (b) channels respectively.

FIG. 5 provides a device of the invention comprising a bundle of cylindrical reactors.

FIG. 6 provides a device of the invention comprising an outer cylindrical tank, with an inlet and an outlet, wherein the cylindrical tank includes several inserted bars bars with LEDs on the walls.

FIG. 7 provides a device of the invention comprising an outer tank, with an inlet and an outlet, wherein the tank includes several inserted reactor bars with LEDs on the walls.

### DETAILED DESCRIPTION OF THE INVENTION

In this invention, a high-intensity ultraviolet light emitting diodes (UV-LED) device and process is provided. This device comes with all the marinization elements to make it able and suitable for subsea, off-shore and for deep and ultra-deep water operation, and also for use with equipment installed either on topside or under water, on the seabed or near the surface for preventing biofouling formation. For example but not limited to, in equipment like membranes, filters and inside pipe systems, and instrumentation or equipment associated with such. The device can be used to protect, for example, any subsea membrane equipment and process involving any pretreatment, MF, UF, NF, RO, IE or resin in different locations. The main objective is to prevent biofouling formation and organic matter deposition/incrustation. In the case of membrane processes, the UV-LED system provides a way to protect equipment and extend membrane life spam without or reduced chemical utilization, is free of maintenance, and avoiding/reducing the very high costs and complexity involved in any subsea maintenance operation.

The device may be used in subsea applications. Hence, it is for use in deep and ultra-deep water operation for preventing biofouling formation in e.g. pipe systems, instrumentation, equipment and membranes wherein a target fluid is involved. The device is hence a subsea biofouling preventer device with means for providing disinfection of a target fluid. The target fluid to be treated is e.g. sea water, water, an oil-water mixture or a gas mixture. In one embodiment, the device may be combined with membrane systems for use in treating sea water for oil field water injection to achieve improved hydrocarbon production.

Recent improvements in UV-LED flux density, stability and life hours have made UV-LEDs a viable solution for replacing traditional UV light sources such as mercury arc lamps, arch lamps, hot and cold cathode lamps and grid lamps. UV LEDs are more environmentally friendly as they do not contain harmful mercury, do not produce ozone and consume less energy. UV energy passes through the cell walls of the microorganism (bacteria, viruses and spores). Once the UV-LED energy is inside the cell, it is absorbed by the DNA, RNA and proteins causing irreversible destruction. Thus, the organism can no longer replicate and is, therefore, no longer infectious. When the microorganism is killed, the basis for biofilm generation and biofouling is removed. The applicant has found that the use of UV-LED is also suitable in offshore and subsea exploration areas. The invention provides an alternative technology for deep and ultra-deep water operation for preventing biofouling formation, and this includes a new device comprising a reactor comprising reactor surfaces, wherein the surfaces comprise high intensity ultraviolet light emitting diodes (UV-LED). The device is for preventing biofouling formation in a subsea system comprising a target fluid, wherein the target fluid runs through the device, being irradiated by the ultraviolet radiation from the UV-LEDs. Sea water disinfection is an effective way to control biofouling, e.g. in membranes. In addition to the direct disinfection by UV radiation of microorganism, the device may further include means for deoxygenation of the target fluid running through the device, e.g. to avoid corrosion and reservoir souring. To achieve the deoxygenation, the device may in one embodiment further include means for oxygen scavenging.

One individual UV-LED unit is too weak in transmitted power to be used alone. Therefore, an assembly with several UV-LEDs is preferably compounded in the reactor to achieve the required dosage and intensity. The rector of the device may have different designs and configurations. One requisite is that ultraviolet radiation from the UV-LEDs transmits through the target fluid to be treated. Preferably, the reactor unit constitutes a chamber, i.e. a LED chamber, designed to treat the target fluid by the ultraviolet rays from the LEDs. In one embodiment, the LED chamber is configured with an inlet and an outlet wherein the target fluid runs through the chamber, i.e. in through the inlet and out through the outlet of the chamber. Preferably, the UV-LEDs provide multidirectional illumination of the target fluid. The UV light in the circulating fluid system makes it an inhospitable environment to microorganisms such as bacteria, viruses, molds and other pathogens present in the fluid to be treated, and hence prevents biofouling formation.

In one embodiment, the device of the invention is designed to provide both disinfection and oxygen scavenging of the target fluid treated by the device. To achieve the oxygen scavenging the device comprises a nanocomposite film that can be activated by ultraviolet illumination complemented by radiation coming from the UVC LEDs of the device. In one embodiment, the disclosed reactor has at least one internal surface coated with a nanocomposite film, activated by UV and illuminated by UV using LEDs. E.g. the reactor has an internal surface coated with a nanocomposite film, such as of a nano crystalline film of titanium dioxide. This is activated by UV radiation, e.g. at 365 nm (UVA) and illuminated by UV 255 nm (UVC) using the LEDs. In this embodiment, the invention provides a multipurpose reactor and process for simultaneous oxygen scavenging and disinfection of a target fluid such as seawater using a nanocomposite film of titanium dioxide activated by ultraviolet illumination complemented by radiation coming from UVC LEDs. The proposed solution is a unique process/configuration that achieves simultaneously oxygen removal and strong disinfection. The invention hence provides an efficient light driven simultaneous oxygen scavenging and strong disinfection device and process in a single unit. Thus, one objective of this invention is to simultaneously prevent corrosion and biofouling formation by removing oxygen and inactivation of microorganisms with a feasible system to be applied at e.g. subsea seabed and at offshore platforms and floating production, storage and offloading units (FPSOs). One objective of this invention is to ensure normal operation, performance and durability of subsea equipment.

In this embodiment, heterogeneous photo-catalysis complemented by UVC radiation is applied for the degradation of organic matter, oxygen scavenging, and disinfection.

UV illumination by the UV LEDs of the device results in the photo-generation of electrons and holes pairs in the conduction and valence band of the TiO₂, respectively. A consequence of the photocatalytic activity is the consumption of molecular oxygen.

Photo-generated holes react with surface hydroxyl groups to generate surface adsorbed hydroxyl radicals (TiOH.+) that oxidize the pollutant to its mineral form. Photo-generated electrons reduce absorbed oxygen to generate superoxide ions (O2.−), which subsequently are reduced to hydrogen peroxide (H₂O₂) and then to water. The intermediate species produced act as a further source of hydroxyl radicals (OH.). OH radicals are capable of killing a wide range of microorganisms; however, the amount by which they are generated, are not enough to produce a sufficient degree of inactivation in practical terms for most real applications. Since UV illumination for photo-catalysis uses UVA light at 365 nm of wavelength, disinfection should be reinforced and complemented by an UVC emitting source that works at 255 nm, a wavelength that offers the highest performance and efficiency for disinfection. During the disinfection process, the UV energy passes through the cell walls of microorganism (bacteria, viruses and spores) and it is absorbed by its DNA and RNA, causing irreversible destruction. Consequently, the device simultaneously eliminates dissolved oxygen and effectively disinfect the target fluid.

The TiO₂ surface irradiated with the appropriate quantity of UVA radiation can produce intermediate species like OH radicals, O₃ and others. In most practical applications, such as membrane protection to prevent biogrowth for topside or working for subsea, Log 4 inactivation is required. Log 1, could allow the growth of biofilm on the photocatalytic surface, causing its final blockage. In the present invention, the device could achieve Log 4 or higher levels of inactivation of microorganisms, simultaneously with a total theoretical oxygen scavenging, because UVC-LEDs are included, that act directly as stronger disinfectants, and use a wavelength of 255 nm.

In one embodiment of the invention, the reactor of the device has an optimized number of UVC-LEDs installed. The reactor is configured such that water, or other target fluid to be treated, passes through the reactor, being irradiated by the UV LEDs, and hence being disinfected. The reactor comprises surfaces, wherein the surfaces comprise high intensity ultraviolet light emitting diodes (UV-LED). The key characteristics of the reactor include reactor configuration, size, UV light intensity, residence time, catalyst surface area and capacity. The length, size and configuration of the reactor are directly proportional to the residence time required to achieve the desired level of disinfection and optional oxygen removal. In one embodiment, the configuration of the reactor of the device is elected from the group of a planar structure, a spiral structure, spiral tubes, concentric tubes, triangular tubes and a cylindrical structure. Configurations in planar, spiral or as concentric tubes can represent a challenge difficult to overcome from the mechanical and operational point of view. Thus, a preferable design is a cylindrical structure. A cylindrical structure complies with the following requirements:

Mechanical simplicity being easier to integrate and assembling into existing pipelines;

Reduced footprint;

Low pressure drop;

Reduced affectation of the characteristics of the fluid;

The possibility of deposition of some encrusting materials or fouling and if this is deposited on a surface is avoided or minimized; and

Actions to facilitate efficient and complete removal are less complex.

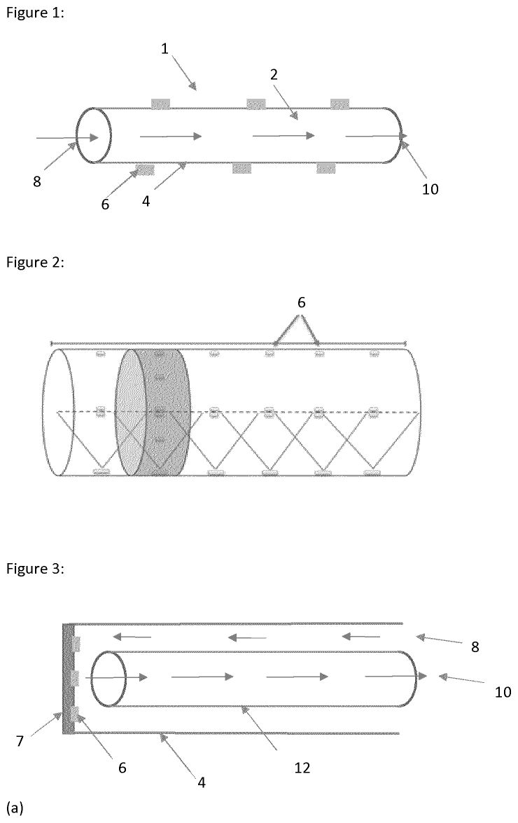

In a preferred embodiment, the reactor has a cylindrical structure, i.e. is a cylinder, wherein an optimized number of UVC-LEDs are installed. In one embodiment, the device has a cylindrical shape wherein this is configured such that water, or other fluid to be treated, passes through the cylinder. E.g. the device has a cylindrical reactor surface where the LEDs are wall mounted and irradiate towards the center. Hence, the cylinder is preferably a right cylinder. Hence, the fluid to be treated enters through a first base and leaves through a second base, wherein the reactor side wall extending between the two bases comprises high intensity ultraviolet light emitting diodes. Such device is shown in FIGS. 1 and 2 wherein a device 1 comprises a cylindrical reactor 2 with walls 4, having wall mounted LEDs 6 irradiating the fluid running through the device through an inlet 8 and out through the outlet 10.

In one embodiment, the UV-LEDs are installed on the outside of the reactor, such as outside the cylinder, i.e. on the outside of the side wall, including all the control and power supply circuit and with a separation between them that maximizes the uniform distribution of the intensity of UV received by the microorganisms in the fluid to be treated. The UV light reaches the fluid that passes through the inner part of the reactor, through a number of windows in the wall, which let pass the majority of the light emitted by the UVC LEDs. In this way, the LEDs do not enter in direct contact with the fluid to be treated.

Alternatively, at least a portion of the LEDs can be integrated in the reactor walls, such as in the cylinder walls. In this case, the UV emitters are encapsulated with a material that isolate and protect them from the radial collapse, such as due to pressure, and from the degradation caused by the seawater and any external agents contained in the water. Hence, in one embodiment at least one surface of the reactor is made of a transparent material to allow UV light passing through. A benefit is that the solution allows the passage of light without or with very little interference. Also, the encapsulation material should be resistant to chemical degradation caused by ultraviolet light, that is, it should not go opaque with the passage of time. The materials, considered suitable as encapsulation layers for the isolated UV-LEDs, independently of the configuration of the reactor chosen, can be selected from the non-limiting group of quartz, acrylic, special silicones, epoxy resin and adhesives. That encapsulation layer could be individual, i.e. for each isolated UV-LED, or could be an integral encapsulation body, i.e. one-piece protecting all LEDs, through the total length of the reactor.

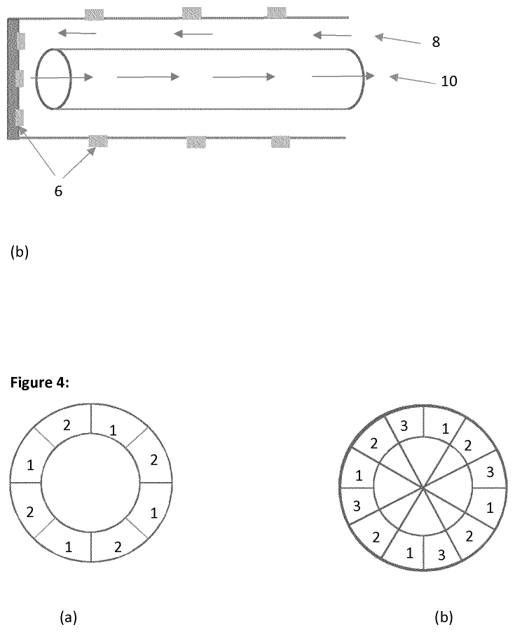

In a further embodiment, the device has a pipe-in-pipe configuration, e.g. wherein one pipe leads the target fluid and the other comprises the UV-LEDs. Such configuration may enable a double pass of the fluid. The device may hence comprise an internal pipe within the outer tank device, wherein surfaces of the reactor unit comprise UV-LEDs. The LEDs may be placed either on surfaces of the internal pipe or on either of the walls of the outer tank. Both the inner pipe and the outer tank may have different configurations. The cross-section of the inner pipe may e.g. be square, rectangular, triangular or circular, and is preferably circular. In a pipe-in-pipe configuration, the inner pipe is e.g. cylindrical and the UV-LEDs are wall-mounted in or on the inner reactor pipe, irradiating outwards towards the target fluid running in the outer pipe. Alternatively, the UV-LEDs are on, or in, the walls of the outer pipe. FIGS. 3a and 3b provide examples of devices of the invention wherein the device has a pipe-in-pipe configuration. In FIG. 3a the LEDs 6 are placed on the side cover 7 and in 3b the LEDs are placed on the reactor walls 4, either on the outer or inner walls, and optionally also on the side cover 7. Hence, a combination of LEDs on the side cover forming the base of the outer tank and on the cylinder walls is suggested. The fluid enters the devices though an inlet 8 and is irradiated by the LEDs 6 and flows out of the outlet 10 of the inner pipe 12.

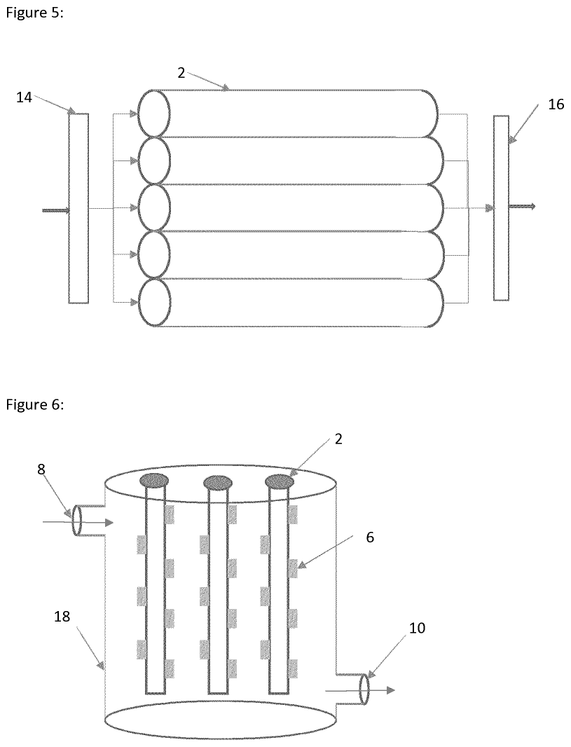

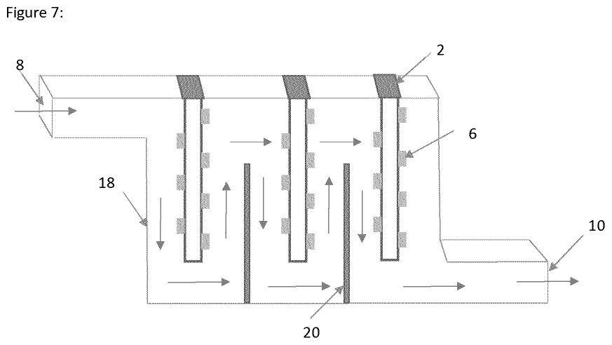

In a further embodiment, several reactors are combined in one device. E.g. several scalable units, for instance such as those shown in FIGS. 3a and 3b, can be replicated and coupled in parallel or in series. To process higher flow capacities several reactors may be combined in parallel, and the device may include a distribution header and/or an outlet header, hence include manifolds, as shown in FIG. 5. In this device five reactors 2 are arranged in parallel coupled to a distribution header 14 and to an outlet header 16. The reactors include LEDs, not shown in the FIG. 5, as earlier disclosed, e.g. arranged around the pipe walls. FIG. 6 shows another example of a device of the invention including several reactors. In the device of FIG. 6 three reactors 2 are inserted in an outer tank 18, wherein a fluid is received through an inlet 8, the fluid is irradiated by LEDs 6 and the tank has an outlet 10. In this device, the tank 18 is cylindrical, but this could as well had any other shape, such as e.g. square. Further, in this configuration the fluid will pass the LEDs one time, and there is no specific flow direction. FIG. 7 shows another example of a device of the invention including several reactors. In the device of FIG. 7 three rectangular reactors 2 are inserted in an outer tank 18, wherein a fluid is received through an inlet 8, the fluid is irradiated by LEDs 6, and the tank has an outlet 10. In this device, the tank 18 is mainly square, and includes internal walls 20 providing a flow specific direction.

In the embodiment wherein the device also is configured to enable deoxygenation, the device comprises internal surfaces coated with a nanocomposite film. Different configurations are possible to ensure that the coating is irradiated by the UV-LEDs of the reactor unit. The coated internal surfaces are elected from the group comprising spheres, discs, walls and bars. The following configurations are suggested:

Cylindrical device filled with spheres coated with a nanocomposite film;

Cylindrical device comprising circular discs coated with a nanocomposite film;

Cylindrical device with an inner wall of cylinder coated with a nanocomposite film;

Cylindrical device with bars coated with a nanocomposite film;

Cylindrical device with rectangular plates coated with a nanocomposite film;

Rectangular device with spheres coated with a nanocomposite film.

Moreover, the same concepts could be applied to other geometric forms. In all the configurations suggested in this embodiment, the target fluid flows through the device and is deoxygenated and disinfected simultaneously, without the need of using chemicals, storage tanks, bulky columns, or other additional equipment. Hence, the devices shown in the Figures could include internal surfaces coated with a nanocomposite film. The device should have adequate internal transfer surface area for the oxygen removal reactions and disinfection by hydroxyl radicals to occur. The area of the coated surface with the nanocomposite film is proportional to the oxygen concentration in seawater. Similarly, the UVA light intensity needed to activate the catalytic surface, i.e. surface coated with the nanocomposite film, is also proportional to the active surface area.

In a further embodiment, the device of the invention may in addition also have surfaces comprising a fouling preventive coating.

The following main identified advantages are associated with the UV-LED device of the invention:

1. Higher life time of the device compared to conventional lamps

2. No rotary or movable parts

3. No chemical use and no chemical waste stream (Very low environmental impact and risk)

4. Low footprint

5. High availability

6. Considerable energy saving compared to disinfection units using conventional lamps.

7. Robustness under pressure

8. Cost reduction by low maintenance frequency

The reactor of the device comprises high-intensity light emitting diodes providing ultraviolet (UV) radiation, hence with a wavelength from 10 nm to 400 nm. More preferably the device uses a wavelength of 200-320 nm, such as short-wavelength ultraviolet light (UVC), also called ultraviolet germicidal radiation (UVGI) with a wavelength of 100-280 nm. Most preferably, the UV-LED device of the invention uses the more effective germicidal UV range (UVC), that is, from 250 nm to 270 nm, with peak in 254 nm of wavelength. The ultraviolet germicidal irradiation (UVGI) kills or inactivates microorganisms by destroying nucleic acids and disrupting their DNA. In one embodiment, to achieve both disinfection and deoxygenation the device may provide a combination of UVA and UVC radiation by LEDs to generate a strong O₂ scavenging and disinfection effect. Hence, the device may include UV-LEDs providing different wavelengths, e.g. such as different sets of UV-LEDs operating at different wavelengths, such as e.g. about 255 nm (for disinfection) and at 365 nm for activation of the composite film.

While there is a concern on heat dissipation for use of UV-LEDs at room temperature, it is possible that the subsea use, wherein sea temperature may be as low as 4° C., can effectively cool down the system. Thus, heat dissipation and refrigeration is not an issue as the complete device will be immersed in seawater at 4° C. Accordingly, the device of the invention is preferably configured with a high resistance against low temperatures.

The reactor comprises high intensity ultraviolet light emitting diodes (UV-LED) included in surfaces of the reactor, such as in either of the walls. These ensure that the fluid operating in the system receives an optimized dosage of UVC radiation by multidirectional illumination. A plurality of UV LEDs are organized on the surfaces of the reactor, preferably evenly distributed, e.g. in a pattern or as a grid. Preferably the LEDs are symmetrically distributed to guarantee uniform intensity. In one embodiment, particularly for a cylindrical device, a configuration using 48 UVC LED's of 6 mW each, located on the internal reflective or no reflective surface of a cylindrical reactor, was found feasible. Also, that configuration, consistent with the simulation results allows to obtain as much as 99.99% of cells inactivation with a resident time less than two seconds. Hence, in one embodiment, the reactor includes 30-80 UV LEDs, such as 40-60 UV LEDs. The number of UV LEDs to include depends on at least the total power needed, how the diodes are distributed and whether all LEDs are to be used at the same time, and also whether a reflective material is used or not.

While encapsulation layer opacity is a concern, degradation/interference on irradiation can also be caused by dirt deposition (salts, suspended solids or others) on the device walls. One way to detect potential fouling is through the measurement of pressure drop between the inlet and outlet of the reactor. For progressive deposition of salts, for example, a sensor may be placed in the reactor for early detection of salt deposits, which could trigger the mechanisms of self-cleaning system. Such means for cleaning may include periodically flush with high flow rate seawater, ultrafiltration permeate, nanofiltration permeate and concentrate, or an acid or base stream generated by other electrochemical devices. A flush with a nanofiltration concentrate may also provide osmotic shock and could help to kill any formed biofilm. Hence, in one embodiment, the device further comprises any of means for pressure measurements, sensors for detection of deposits and means for self-cleaning. Further, in one embodiment, the device includes an UV detection system to evaluate overall system performance.

It is expected that the durability of the LEDs be significantly higher than conventional lamps. Further, other possibilities to extend lifetime of the equipment, applicable to the disclosed solution would be to design the device with redundancy on UV-LED units. Not all of them would then be used at once, but would rather have an alternate operation, and when one unit fails there would be reserve units to be turned on. That would be simpler than designing lamp modules to be replaced by remotely operated underwater vehicles (ROV), with all the complexity that this involves. To achieve required redundancy, the LED chamber may have the LEDs grouped in two or more channels that can be individually controlled, and powered. Each channel could have its own sensors, such as sensing capability to tell that the correspondent channel is lit or working properly. Sensing capabilities include photodiode or another photo sensitive electronic device for the desired wavelength. This type of sensors can detect loss of light due to either LED problems, or light path blockade due to scaling for example. Other potential sensing capabilities perform current measurements for each channel, and hence provide an indirect indication of problems with the LEDs. In one embodiment, the individual LED arrangement is such that the channels are interwoven with one another. This makes possible that in case a channel must be turned off while other is turned on, the same amount of lighting power is delivered to the same volume, and the light paths and geometry also remains the same. For a cylindrical reactor surface, such as shown in FIGS. 1 and 2, where the LEDs are wall mounted and irradiate towards the center, this can be arranged with two or three channels as shown in FIGS. 4a and 4b. Hence, the LEDs can be arranged in 2, 3 or more sets of channels running in parallel in the side wall from the inlet to the outlet, wherein each channel has a number of LEDs. In the case where the LEDs are arranged in a planar grid, these can be arranged with 2 or 3 channels. Inside a channel, a single LED or a cluster of LEDs can be placed, depending on the desired total power irradiated per unity of area. The fault tolerance of the whole system is improved with increasing number of channels. As channels are lost due to wear or loss of lighting elements, other channels can be activated, eliminating the need of intervention to swap modules or remove the whole device.

For the device of the invention, the minimum flow velocity through the reactor is about 1 m/s, such as at least 2 m/s. That is to prevent salt deposition in the internal surface of the unit. UV-LED disinfection could be performed at high flow velocity without impact in efficiency. Alternatively, auto self-cleaning mechanism would also be an option. As the device is intended to fit with standard pipes conducting the water or target fluid, it preferably has dimensions corresponding to dimensions of standard pipes. In one embodiment, wherein the reactor has a cylindrical structure, this has a diameter of 1-100 centimeters, such as 10-80 centimeters, and e.g. around 30. In a small-scale testing equipment (prototypes) the size can be smaller, e.g. with a device diameter of 2-8 centimeters. In the example prototype, the diameter of the cylindrical reactor is 5.56 centimeters.

Reference is made to Example 1 wherein the disinfection of 0.072 m³/h of real seawater has been analyzed. Based on the defined diameter of the cylindrical reactor as 5.56 cm and knowledge about the concentration of microorganisms in seawater the calculation of the amount and power of the UVC LEDs needed has been made. From this analysis and calculation the following has been found:

The reactor prototype may have a volume of 0.5-4.0 cm³, such as around 2.0 cm³. With a diameter of 5.56 cm, a length of 8.0 cm, the volume is 1.94 cm³. In full-scale equipment, with a cylindrical device with a bigger diameter also the volume will be bigger.

As shown in the first part of Example 1, the necessary UV radiation intensity having a real germicidal effect measured using a radiometer was 15.5 W/m². Also, as the germicidal UV radiation is the UVC type and the efficiency of that comparison was only 8.69%, it was thus found that the net or effective dosage to achieve 99.99% of disinfection was 136.5 J/m². In one embodiment, the device provides a radiation dosage of 50-300 J/m², more preferably, 100-200 J/m² and most preferably around 140 J/m². Based on the dimensions given above, and UV radiation intensity needed, the total power needed is around 217 mW. The calculation is provided in Example 1. In one embodiment, the device is dimensioned to provide a total power from the UV LEDs of at least 100 mW, such as at least 200 mW, such as 200-500 mW. Eg. with an efficiency of 75.5% and 6 mW of power each, the total power and number of UV LEDs needed is 288 mW and 48 units.

The device of the invention is designed for subsea use, and hence must withstand the high pressure at depths e.g. of 3000 meter (i.e. 300 bars). The challenge about pressure compensation is essentially structural. Compensation must be granted such that a pressure differential of 300 bars from the outside to the inside does not collapse the device. For a device with a cylindrical reactor the challenge is to protect the structure from radial collapse and at same time to grant passage for the UV radiation. Such protection could be achieved with transparent materials, with high mechanical resistance to pressure, and chemical resistance to degradation caused by UV light. In this invention, we are considering several applicable options for that material, including but not limited to, quartz, acrylic, silicone, epoxy resin and adhesives, among others. While the encapsulation layer strength is directly proportional to the thickness, the light intensity is inversely proportional, as the layer will act as a filter. As previously mentioned, that encapsulation layer could be individual (for each isolated UV-LED) or an integral encapsulation body (one-piece protecting all LEDs) through the total length of the unit. A planar structure is possible in the case of LED lamps. In this case encapsulation layer with any material of previously mentioned is also considered, with same challenges imposed as with cylindrical structures. The thickness will grow proportionally to the area covered.

In addition to that the reactor should have a high mechanical resistance to pressure, and a chemical resistance to degradation, it has been found that a reflective material utilized in the wall of the reactor can significantly increase the UV dose for the same number of LEDs. For instance, it has been found that by changing from a non-reflective material to a reflective material the UV intensity can be increased by a factor of about 3. For example by using the UVC-LED device of the invention, the dosage, e.g. of about 140 J/m², the target cells inactivation can be achieved in 1.95 s and 0.62 s of resident time without or with reflective material, respectively. Hence, if using a reflective material on the inside of the reactor walls the flow velocity can be increased. In one embodiment, at least parts of the inner walls of the reactor comprise a reflective material. E.g. the inner walls, may have a reflective surface material. The reflective material is e.g. selected from the group of any reflective material resistant to seawater, and this may include e.g. polytetrafluoroethylene (PTFE), polypropylene (PP), polyethylene (PE) and others. Therefore, as shown above, the optimized distribution of LED sources combined with a reflective material increases the efficiency of the dose and the disinfection for the same reactor size, and hence it reduces dramatically the residence time.

The device disclosed may be inserted and/or coupled into a pipe, or other part of a system equipment, conducting the target fluid, such as through a flange or other mechanism, to perform its function as a section or stretch of the pipe itself. In other embodiments, other arrangements or configurations including the device are possible. The device may be included in systems comprising membranes, filters and inside pipe systems, instrumentation or equipment associated with any of these. The device can be used to protect, for example, any subsea membrane separating process involving any pretreatment, microfiltration (MF), ultrafiltration (UF), nanofiltration (NF), reverse osmosis (RO), ion exchange (IE) or resin in different locations. Further, the device may be used with equipment installed either on topside or under water, on the seabed or near the surface, and with associated methods of treating seawater for oil field water injection. The main objective is to prevent biofouling formation and organic matter deposition/incrustation in an any parts of the system.

Further, the disclosed UV-LED disinfection device could occupy, for example, different positions or locations within a subsea treatment system, depending on the specific needs of the process and the equipment involved. Such system could for example be designed for sulphate removal for water injection, low salinity water for flooding, oil-water separation or gas separation. Such system could include one or several of the devices of the invention, such as 1-5, or 1-3 UV-LED devices. Hence, the device is configured to match or include means for integration or coupling to other equipment. Hence, the device of the invention may be combined with equipment and parts for separation processes, including e.g. membrane modules and pumps, e.g. including parts for underwater coarse filter (CF), media filter (MF), microfiltration (MF), ultrafiltration (UF), nanofiltration (NF), reverse osmosis (RO), ion exchange (IE), electrodialysis, gas separation or de-piling.

In addition to the feasibility of UVC-LED disinfection effectivity for seawater, the seawater turbidity has an influence on the final level of cells inactivation. Turbidity is much higher for unfiltered water than for filtered water, and it has been found that turbidity dramatically reduces the efficiency of UV disinfection. Accordingly, the device is preferably combined with a pre-filter, to provide a pre-treatment step, to ensure full efficiency. Hence, the UVC-LED device is preferably positioned after a pre-filter unit.

In one embodiment, at least one UV-LED device is included in a system comprising the units listed in the different systems below, wherein the UV-LED devices can be included in any positions:

1. Pre-treatment filter; ultrafiltration membrane; nanofiltration membrane

2. Microfiltration membrane; ultrafiltration membrane; reverse osmosis

3. Microfiltration membrane; ultrafiltration membrane; electrodialysis

4. Pre-treatment filter; gas separation membrane

5. Microfiltration membrane; De-piling membrane

Preferably, systems 1-3 above are for seawater treatment, while system 4 is meant for gas treatment and system 5 is for an oil-water mixture treatment. In one embodiment, the system includes an UV-LED device positioned before an equipment including a membrane for filtration, providing a way to extend membrane life spam.

In a further aspect, the invention provides a process for subsea operation of a target fluid in a system, comprising a step wherein high intensity ultraviolet radiation from UV-LEDs transmits through the target fluid to be treated to prevent biofouling formation in any parts of the system. In the process, a device as disclosed in the first aspect, is used. The embodiments and features described in the context of the first aspect of the present disclosure also apply to this second aspect. In one embodiment, the process provides multidirectional illumination of the target fluid. Preferably, the UV radiation is of the UVC type and the radiation provides an effective dosage to achieve at least 70% disinfection, such as at least 80%, preferably at least 90%, and even as much as 99% of disinfection, such as 99.99% of disinfection. In one embodiment, the process is part of a separation process. Further, in one embodiment the process provides a step of disinfection and a step of oxygen scavenging achieved by the device of the first aspect, wherein the device comprises internal surfaces coated with a nanocomposite film, activatable by UV radiation, and preferably wherein the steps take place simultaneously.

### EXAMPLES

The present disclosure may be further described by the following non-limiting examples.

### Example 1: Calculation of Power Needed from UV-LEDs for Seawater Disinfection

The calculation of the amount and power of the UVC LEDs needed are based on a seawater disinfection experiment performed using a conventional UV disinfection unit of 15 Watts and a flow rate of 0.50 m³/h of real seawater from the Guanabara Bay at Rio de Janeiro. Table 1 details the main disinfection results and characteristics of the seawater from Guanabara Bay.

TABLE 1

| | | | Total | | | |
|---|---|---|---|---|---|---|
| | | | aerobic | | Residence | |
| Flow rate | Conductivity | Turbidity | bacteria | Log of | time | Intensity |
| (m³/h) | (mS/cm) | (NTU) | (UFC/ml) | inactivation | (s) | (W/m²) |
| Feed | 46.63 | 6.1 | 3.88E+05 | — | | |
| (seawater) | | | | | | |
| 0.498 | 43.53 | 2 | 6 | 4.81 | 8.81 | 178.317 |
| 0.517 | 43.53 | 2.1 | 2 | 5.29 | 8.47 | |
| 0.508 | 45.3 | 2.2 | 3 | 5.11 | 8.63 | |
| 0.482 | 45.3 | 2.2 | 3 | 5.11 | 9.08 | |
| 0.419 | 47.11 | 2.6 | 0 | Complete | 10.45 | |
| | | | | inactivation | | |
| 0.394 | 47.11 | 2.6 | 0 | Complete | 11.12 | |
| | | | | inactivation | | |
| 0.182 | 47.33 | 2 | 1 | 5.59 | 24.02 | |
| 0.185 | 47.33 | 2 | 0 | Complete | 23.69 | |
| | | | | inactivation | | |
| 0.072 | 47.8 | 2.1 | 0 | Complete | 61.20 | |
| | | | | inactivation | | |
| 0.074 | 47.25 | 2.1 | 0 | Complete | 59.07 | |
| | | | | inactivation | | |

Per this experiment, using conventional UV lamps, in the entire UV spectrum (UVA+UVB+UVC etc), the total intensity at a flow rate of 0.5 m³/h was 178.32 W/m², the resident time was 8.8 s, and the total dosage to obtain Log 4.8 (+99.998%) of cells inactivation was 1569 J/m²; however, when the intensity of UVC radiation, that is the type of radiation which have a real germicidal effect was measured using a radiometer, the results was only 15.5 W/m².

Accordingly, the useful UV radiation intensity was 15.5 W/m², and consequently the efficiency was as low as 8.69%. Also, as the germicidal UV radiation is the UVC type and the efficiency was only 8.69%. Thus, the net or effective dosage was 136.5 J/m² only.

Then, for the case of 0.072 m³/h of seawater, with the equations:

D=I×t (1)

I=P/A (2)

A=πrh (3)

V=π2h (4)

Where,

D=Dosage to inactivate a desired number of microorganisms in J/m²

I=UVC light intensity in W/m2

t=Residence time in seconds

P=Total power in Watts that is needed

h=Usable length of the reactor

A=Contact area in m²

r=Internal radius of the reactor in meters

V=Volume of the reactor in m³

It is found that:

Volume of the reactor needed is 1.94×10⁻⁴ m³, and from there h=0.08 m.

Also, A=0.014 m² and from here with I=P/A, it is found that the total power that is needed is around 217 mW. Considering the use of UVC LED, with an efficiency of 75.5% and 6 mW of power each, the total power and number of UVC LED's needed is 288 mW and 48 units respectively.

### Example 2: Testing of UVC-LED Flow Cells

Initial experimental tests using an UVC-LEDs flow cell have been performed. The tests were conducted in a UVC-LED reactor based on a simple design as disclosed in Example 1 with not optimized distribution of LED light sources. The results, gave very good disinfection effect of microorganisms, and hence proof the technical feasibility of the disclosed concept. The results also demonstrated that there is space for improvement on design, and indicated that the number of LED units could be minimized and how to reduce the residence time. In addition to show the feasibility of UVC-LED disinfection effectivity for seawater, the results show the importance and influence of seawater turbidity on the final level of cells inactivation. For the filtered seawater (with an 80 μm pre-filter) the turbidity was below 1 NTU (Nephelometric Turbidity Unit), and the cell inactivation was around 99.99% (Log 4), compared with 90% inactivation when unfiltered water was used (5 NTU). So definitely, the increase in turbidity dramatically reduces the efficiency of UV disinfection. But in any case, the reductions for tests performed at greater than 4 NTU turbidity were always close to 90%, that is, Log 1. The result shows that the disinfection system benefits from a pre-filter to ensure their full efficiency, in that way the UVC-LED unit should preferably be positioned after a filter unit.

For the filtered sea water, and hence low turbidity, an efficiency of more than 99.99% was obtained from 5.3 s to 3.1 s of residence time. For higher flow rates, i.e. less than 3 s of residence time, still 99.95% cell inactivation was obtained.

1. A device for preventing biofouling formation in a subsea system comprising a target fluid, the device comprising a reactor comprising reactor surfaces, wherein the surfaces comprise high intensity ultraviolet light emitting diodes (UV-LED).

2. A device as claimed in claim 1, wherein an assembly of several UV-LEDs are compounded in the reactor surfaces and the UV-LEDs are configured to transmit radiation through the target fluid to disinfect this.

3. A device as claimed in claim 1, wherein the configuration of the reactor is elected from the group of a planar structure, a spiral structure, spiral tubes, concentric tubes, triangular pipes and a cylindrical structure, and preferably is a cylindrical structure.

4. A device as claimed in claim 1, wherein the reactor constitutes a LED chamber with an inlet and an outlet wherein the target fluid runs through the chamber and is treated by the ultraviolet rays.

5. A device as claimed in claim 1, wherein at least one surface comprises transparent material to allow UV light to pass through.

6. A device as claimed in claim 1, wherein at least one reactor surface comprises a fouling preventing coating.

7. A device as claimed in claim 1, wherein the device has a pipe-in-pipe configuration.

8. A device as claimed in claim 1 further comprising internal surfaces coated with a nanocomposite film for enabling deoxygenation.

9. A device as claimed in claim 8 wherein the nanocomposite film comprises titanium dioxide.

10. A device as claimed in claim 1 wherein the UV-LEDs are grouped in two or more channels that can be individually controlled and powered.

11. A device as claimed in claim 1 wherein the device further comprises any of means for pressure measurements, sensors for detection of deposits, means for self-cleaning and an UV detection system to evaluate overall system performance.

12. A device as claimed in claim 1 wherein the device is inserted or coupled into a pipe, or other part, of a system conducting the target fluid, to perform its function on the target fluid to prevent biofouling in the system.

13. A device as claimed in claim 12 comprising means for coupling to or integration to equipment and parts for separation processes like microfiltration (MF), ultrafiltration (UF), nanofiltration (NF), reverse osmosis (RO), ion exchange (IE), electrodialysis, gas separation or de-piling.

14. A process for subsea operation of a target fluid, comprising a step wherein the target fluid runs through a device as claimed in claim 1 wherein high intensity ultraviolet radiation from UV-LEDs transmits through the target fluid to prevent biofouling formation in any parts of a system wherein the device takes parts.

15. A process as claimed in claim 14 comprising a simultaneous step of disinfection and deoxygenation.
